(19) United States
(12) Patent Application Publication
(10) Pub. No.: US 2020/0326338 A1
(43) Pub. Date: Oct. 15, 2020

(54) DETECTION AGENT FOR BIOASSAY AND SIGNAL AMPLIFICATION METHOD USING SAME

(71) Applicant: TANAKA KIKINZOKU KOGYO K.K., Tokyo (JP)

(72) Inventors: Shinichi Kato, Kanagawa (JP); Daisuke Ito, Kanagawa (JP); Soichiro Sekine, Kanagawa (JP)

(73) Assignee: TANAKA KIKINZOKU KOGYO K.K., Tokyo (JP)

(21) Appl. No.: 16/760,081

(22) PCT Filed: Oct. 31, 2018
(86) PCT No.: PCT/JP2018/040435

§ 371 Date: Apr. 29, 2020

(30) Foreign Application Priority Data
Oct. 31, 2017 (JP) 2017-210952

(51) Int. Cl. G01N 33/553

(52) U.S. Cl. CPC G01N 33/553; B82Y 40/00

(57) ABSTRACT

The present invention provides, in a method for measuring a subject substance by using a binding partner that specifically recognizes a substance to be measured, a detection agent for amplifying a signal from a reporter substance that indirectly binds to the substance to be measured via the binding partner, and a signal amplification method using the detection agent. The present invention pertains to the detection agent in which the binding partner which specifically recognizes a substance to be measured is immobilized on gold nanoparticles together with a plurality of the reporter substances.

### TECHNICAL FIELD

The present invention relates to a detection agent for amplifying a signal and a measurement method using the detection agent, in a method for measuring a subject substance by using a biologically specific binding partner.

### BACKGROUND ART

A method for measuring a subject substance using binding by a biologically specific binding partner represented by an antigen-antibody reaction can very specifically measure even only a very small amount of a substance to be measured, with high sensitivity, and quickly and easily, since the binding partner has high specificity and high binding affinity to the substance to be measured. Therefore, immunoassay, one of the assays using a biologically specific binding partner, is not only used in testing of measurement items such as hormones, tumor markers, viruses and bacteria, autoantibodies, blood coagulation and fibrinolysis systems in the field of clinical chemistry testing, but also currently applied to testing in a wide range of fields, such as detection of harmful substances contained in food, investigation of environmental hormones (exogenous endocrine disruptors) contained in soil and rivers, and screening for drugs of abuse.

There are several variations in the reaction format of the immunoassay. These can be classified from the viewpoint of measurement principle, that is, from the viewpoint of (1) whether the reaction between a substance to be measured and an antigen or antibody is competitive or non-competitive, (2) whether or not B/F (Bound/Free) separation is required, and (3) whether or not labeling with a reporter substance is required. Among the immunoassays, heterogeneous labeled immunoassays, which require B/F separation by labeling an antigen or an antibody with some reporter substance, are important measurement methods with high versatility. The methods are further classified into competitive methods and non-competitive methods. In the competitive method, for example, when the substance to be measured is an antigen, a certain amount of the antigen is immobilized on a solid phase, and an antibody specific thereto is labeled with a reporter substance. When an antigen to be measured and a limited amount of labeled antibody are added to the immobilized antigen, the immobilized antigen and free antigen react competitively with the antibody, and as the amount of free antigen increases, the amount of labeled antibody adsorbed to the solid phase decreases. By changing the amount of free antigen and measuring signal intensity from the labeled antibody remaining on the solid phase, a standard curve (dose-effect curve) can be created. The amount of the antigen to be measured can be determined by performing a competitive reaction with the immobilized antigen for the antigen to be measured whose concentration is unknown and inserting it into the standard curve. On the other hand, in the non-competitive method, when the substance to be measured is an antigen, the antigen is reacted with an excessive amount of the labeled antibody, and the amount of an immune complex quantitatively produced is determined based on the signal intensity from the labeled antibody.

In the non-competitive method, the reaction quickly reaches equilibrium by using excess antibody, and a trace amount of antigen can be efficiently converted to signal intensity. Therefore, when the non-competitive method is adopted, the analysis time can be easily reduced, the measurement accuracy can be increased, and higher sensitivity can be obtained. In particular, an assay in which a solid phase immobilized with a certain excess amount of antibody, the antigen to be measured is captured on the solid phase, and a labeled antibody that recognizes a different antigenic determinant on an antigen molecule is added in excess to react is called a sandwich assay because an antigen forms a complex sandwiched between two types of antibodies. Sandwich assays are generally highly sensitive and are currently most commonly used as highly sensitive assays for protein antigens.

In heterogeneous labeled immunoassays, especially sandwich assays, further improvement in sensitivity is required to further shorten the analysis time and to develop new measurement items. In the labeled immunoassay, since the amount of the antigen to be measured correlates with the signal intensity from the labeled antibody bound thereto, it is necessary to enhance the signal from the labeled antibody in order to improve the sensitivity of the assay. Therefore, with respect to reporter substances for labeling antibodies, various types of reporter substances and methods for detecting a signal finally generated have been studied to date. The most frequently used reporter substances at present are enzymes, and typical examples of which include horseradish peroxidase (HRP), β-galactosidase (β-GAL), alkaline phosphatase (ALP), and the like. By reacting these enzymes with an appropriate substrate, a dye, a fluorescent substance, a luminescent substance or the like is finally produced, and change in absorbance due to the product, or a fluorescence intensity or a luminescence intensity is measured as a signal. The advantage of using an enzyme is that it is possible to amplify the signal finally generated since the amount of the dye, the fluorescent substance or the luminescent substance can be increased by enzymatic activity, as compared with the case where the dye, the fluorescent substance or the luminescent substance itself is used as the reporter substance. In particular, in a method of using an enzyme such as HRP, β-GAL, ALP or luciferase as the reporter substance, adding luminol, an adamantyl dioxetane derivative, luciferin or the like as a substrate, and detecting light emitted by a reaction product (chemiluminescent enzyme immunoassay), it is possible to obtain higher sensitivity than when using a radioisotope as the reporter substance.

However, despite the study of the reporter substances and their detection methods as described above, detection sensitivity of the current labeled immunoassay is not satisfactory, and there is still room for improvement in the method for amplifying the signal from the labeled antibody. Substances to be measured by the labeled immunoassay contain many small molecules and peptides such as hormones and tumor markers, and the number of labeled antibodies that can bind to these small molecules is limited. In particular, in sandwich assays, since these substances are captured by an antibody immobilized on a solid phase, generally only one labeled antibody can bind to a substance to be measured. Also, the number of reporter substances that can be bound to the labeled antibody is limited to about 1 to several. Although the amount of the substance to be measured correlates with the signal intensity from the labeled antibody bound thereto, the number of reporter substances that can indirectly bind to the substance to be measured via the antibody is limited, and this has been a factor that has hindered improvement in sensitivity in labeled immunoassays.

In order to increase the number of reporter substances that indirectly bind to the substance to be measured and amplify the signal intensity correlated with the amount of the substance to be measured, it has been proposed to use an antibody that specifically recognizes the substance to be measured immobilized on a detection support such as beads together with detectable moieties (Patent Literature 1). There, any one of the plurality of antibodies immobilized on the detection support binds to the substance to be measured captured by the antibody immobilized on a capture support to form a sandwich-type immune complex. By further reacting an antibody on the detection support that is not involved in binding to the substance to be measured with a binder having a detectable part that generates a signal, many detectable parts can be finally indirectly bound to one substance to be measured, and it is possible to amplify the signal intensity. The detectable part can be bound to the detection support via a binder as described above, or can be directly immobilized on the detection support together with a plurality of antibodies, or the detectable part is previously bound to a plurality of antibodies, which can be immobilized on the detection support. Polystyrene beads are disclosed as the detection support, and polystyrene beads with a size of about 0.1 to 50 μm, and particularly about 1 to 3 μm in diameter are described. A key factor for amplifying the signal is the size of the surface area of the detection support to which the antibody can bind, and it is also disclosed that the larger the surface area of the detection support, the higher the signal amplification rate. However, it is not described that nanoparticles having a diameter of about several nm to several tens of nm can be used as the detection support. In particular, it is not disclosed that metal nanoparticles such as gold nanoparticles are used as the detection support.

On the other hand, Patent Literature 2 discloses gold nanoparticles with an antibody and an enzyme immobilized on the surface thereof. There, in order to avoid denaturation and inactivation of the antibody and deterioration of function of the antibody without exposing an antigen binding site due to random orientation by physically adsorbing the antibody to the gold nanoparticles, it has been proposed to immobilize the antibody on the gold nanoparticles via an antibody binding protein-gold binding peptide. However, despite the use of antibody binding protein and gold-binding peptide to immobilize the antibody on the surface of gold nanoparticles with appropriate orientation, it was difficult to amplify the signal of immunoassay using gold nanoparticles immobilized with an enzyme-labeled antibody. Patent Literature 2 describes that an antigen was adsorbed on a plate using an antigen solution at a concentration of 1 mg/ml, and this was detected by gold nanoparticles immobilized with an antibody and a labeled antibody, and discloses that the amount of labeled enzyme that could bind to the antigen through the gold nanoparticles to form an immune complex was by no means sufficient, and required time as long as 90 minutes even using standard substrates and colorimetric methods to detect the signal from the labeled enzyme.

### CITATION LIST

### Patent Literature

Patent Literature 1: JP 2015-508180 W
Patent Literature 2: JP 2013-151454 A

### SUMMARY OF INVENTION

### Technical Problem

The present invention provides, in a method for measuring a subject substance by using a binding partner that specifically recognizes a substance to be measured, a method for amplifying a signal from a reporter substance that indirectly binds to the substance to be measured via the binding partner. Moreover, the present invention provides a detection agent that is used in a method for measuring a subject substance by using a binding partner that specifically recognizes a substance to be measured, and can bind to the substance to be measured via the binding partner and amplify a signal from a reporter substance. Further, the present invention provides a method for improving sensitivity of measurement and a detection agent used therefor, in a method for measuring a subject substance by using a biologically specific binding partner labeled with a reporter substance.

### Solution to Problem

In order to further improve measurement sensitivity in a method for measuring a subject substance by using a biologically specific binding partner labeled with a reporter substance, the present inventors have studied methods for amplifying a signal from the reporter substance. As a result of intensive studies by the present inventors, it has been found that a binding partner that specifically recognizes a substance to be measured is directly immobilized on gold nanoparticles together with a plurality of reporter substances, and this is reacted with the subject substance, whereby the number of reporter substances that indirectly bind to the substance to be measured can be markedly increased, and a signal from the reporter substance correlated with the amount of the substance to be measured can be significantly amplified.

That is, the present invention relates to the following (1) to (36).

(1) A method for measuring a subject substance, including:

(i) forming a complex containing a substance to be measured, and a detection agent composed of a first binding partner that specifically recognizes the substance to be measured, a plurality of reporter substances and gold nanoparticles, and

(ii) measuring signals from the reporter substances contained in the complex, wherein

the first binding partner is directly immobilized on the gold nanoparticles in the detection agent.

(2) The method according to (1) above, wherein the complex further contains a second binding partner that specifically recognizes the substance to be measured, which is immobilized on a solid support.

(3) The method according to (1) above, wherein the complex contains the substance to be measured immobilized on a solid support.

(4) The method according to (1) above, wherein

the (i) is a process of contacting a liquid sample containing the substance to be measured, with the detection agent composed of a first binding partner that specifically recognizes the substance to be measured, a plurality of reporter substances and gold nanoparticles to form a complex containing the substance to be measured and the detection agent.

(5) The method according to (4) above, wherein

the process (i), simultaneously with or after the contact, further includes a process of contacting a solid support on which a second binding partner that specifically recognizes a substance to be measured is previously immobilized, and

the (ii) is a process of measuring signals from the reporter substances contained in the complex formed on the solid support.

(6) The method according to (4) above, wherein

the process (i) is a process performed in a reaction system containing a solid support on which the substance to be measured is previously immobilized, and

the (ii) is a process of measuring signals from the reporter substances contained in the complex formed on the solid support.

(7) The method according to (1) above, wherein

the (i) is a process of using at least a liquid sample containing the substance to be measured, the detection agent composed of the first binding partner that specifically recognizes the substance to be measured, a plurality of reporter substances and gold nanoparticles, and a solid support on which a second binding partner that specifically recognizes the substance to be measured is previously immobilized, and is

a process of forming a complex containing the substance to be measured and the detection agent by contacting the solid support on which the second binding partner is previously immobilized, with the liquid sample containing the substance to be measured, subsequently contacting the detection agent composed of the first binding partner that specifically recognizes the substance to be measured and the plurality of reporter substances and the gold nanoparticles, and

the (ii) is a process of measuring signals from the reporter substances contained in the complex formed on the solid support.

(8) The method according to any of (2), (3), and (5) to (7) above, wherein the solid support is selected from the group consisting of a microplate, a magnetic particle, a porous membrane, and a microfluidic chip.

(9) The method according to (8) above, wherein the solid support is a magnetic particle.

(10) The method according to (9) above, wherein the magnetic particles have an average particle diameter of 0.3 to 3 μm.

(11) The method according to any of (1) to (10) above, wherein the gold nanoparticles have an average particle diameter of 20 to 150 nm.

(12) The method according to any of (1) to (11) above, wherein the binding partner is an antigen or an antibody or an antigen-binding fragment thereof.

(13) The method according to (12) above, wherein the binding partner is an antibody or an antigen-binding fragment thereof.

(14) The method according to any of (1) to (13) above, wherein the reporter substance is selected from the group consisting of a radioisotope, an enzyme, a fluorescent substance, and a luminescent substance.

(15) The method according to any of (1) to (13) above, wherein the reporter substance is an electrochemically active luminescent substance or an enzyme that generates an electrochemically active substance as a reaction product.

(16) The method according to any of (4) to (15) above, wherein the liquid sample is a biological fluid.

(17) A detection agent for measuring a subject substance, composed of

a first binding partner that specifically recognizes a substance to be measured, a plurality of reporter substances and gold nanoparticles, wherein

the first binding partner is directly immobilized on the gold nanoparticle,

the reporter substance is directly immobilized on the first binding partner or the gold nanoparticle, and

the reporter substance can generate a signal with an intensity correlated with the amount of the substance to be measured bound to the first binding partner.

(18) The detection agent according to (17) above, wherein the gold nanoparticles have an average particle diameter of 20 to 150 nm.

(19) The detection agent according to (17) or (18) above, wherein the binding partner is an antigen or an antibody or an antigen-binding fragment thereof.

(20) The detection agent according to (19) above, wherein the binding partner is an antibody or an antigen-binding fragment thereof.

(21) The detection agent according to any of (17) to (20) above, wherein the reporter substance is selected from the group consisting of a radioisotope, an enzyme, a fluorescent substance, and a luminescent substance.

(22) The detection agent according to any of (17) to (20) above, wherein the reporter substance is an electrochemically active luminescent substance or an enzyme that generates an electrochemically active substance as a reaction product.

(23) The detection agent according to any of (17) to (22) above, wherein the reporter substance is directly immobilized on the first binding partner.

(24) A kit for measuring a subject substance, including a device composed of the detection agent as defined in any of (17) to (23) above, and a solid support including a complex forming part, wherein

the complex forming part of the device is a part on which a second binding partner that specifically recognizes a substance to be measured or the substance to be measured is immobilized.

(25) A kit for measuring a subject substance, including

a device composed of the detection agent as defined in any of (17) to (23) above, a complex forming agent holding part, and a solid support including a complex capturing part, wherein

the complex forming agent holding part of the device contains a complex forming agent composed of magnetic particles on which a second binding partner that specifically recognizes a substance to be measured or the substance to be measured is immobilized, and

the complex capturing part of the device includes a mechanism that captures the complex forming agent by a magnetic field being applied.

(26) A kit for measuring a subject substance, including

a device composed of the detection agent as defined in any of (17) to (23) above, a complex forming agent, and a solid phase support including a complex capturing part, wherein

the complex forming agent is composed of magnetic particles on which a second binding partner that specifically recognizes a substance to be measured or the substance to be measured is immobilized, and

the complex capturing part of the device includes a mechanism that captures the complex forming agent by a magnetic field being applied.

(27) The kit according to (24) above, wherein the solid support is selected from the group consisting of a microplate, a magnetic particle, a porous membrane, and a microfluidic chip.

(28) The kit according to (25) or (26) above, wherein the solid support is a microfluidic chip.

(29) The kit according to any of (25) to (28) above, wherein the magnetic particles have an average particle diameter of 0.3 to 3 μm.

(30) An immunoassay system containing the kit as defined in any of (24) to (29) above, and a measurement apparatus including a device mounting part to which the device included in the kit can be attached and detached and a signal detection part that can measure a signal generated from the reporter substance of the detection agent included in the kit.

(31) A device for measuring a subject substance, including

a detection agent holding part, and a solid support including a complex forming part, wherein

the detection agent holding part contains the detection agent as defined in any of (17) to (23) above, and

the complex forming part is a part on which a second binding partner that specifically recognizes a substance to be measured or the substance to be measured is immobilized.

(32) A device for measuring a subject substance, including

a detection agent holding part, a complex forming agent holding part, and a solid support including a complex capturing part, wherein

the detection agent holding part contains the detection agent as defined in any of (17) to (23),

the complex forming agent holding part contains a complex forming agent composed of magnetic particles on which a second binding partner that specifically recognizes a substance to be measured or the substance to be measured is immobilized, and

the complex capturing part includes a mechanism that captures the complex forming agent by a magnetic field being applied.

(33) The device according to (31) above, wherein the solid support is a porous membrane or a microfluidic chip.

(34) The device according to (32) above, wherein the solid support is a microfluidic chip.

(35) The device according to (32) or (34) above, wherein the magnetic particles have an average particle diameter of 0.3 to 3 μm.

(36) An immunoassay system containing the device as defined in any of (31) to (35) above, and a measurement apparatus including a device mounting part to which the device can be attached and detached and a signal detection part that can detect a signal generated from the reporter substance of the detection agent included in the detection agent holding part of the device.

### Advantageous Effects of Invention

The present invention provides a method for improving sensitivity of measurement and a detection agent used therefor, in a method for measuring a subject substance by using a biologically specific binding partner labeled with a reporter substance.

In conventional labeled immunoassay, for example, the number of enzymes that can bind to an antigen by binding an enzyme-labeled antibody to the antigen is generally limited to about one, thus there has been a limit to amplify a signal from the enzyme bound to the antigen to increase the sensitivity of measurement. However, in the present invention, a detection agent in which a binding partner that specifically recognizes a substance to be measured is immobilized on gold nanoparticles together with a plurality of reporter substances is provided, whereby the number of reporter substances that indirectly bind to the substance to be measured can be markedly increased, and a signal intensity from the reporter substance correlated with the amount of the substance to be measured can be significantly amplified.

Further, the detection agent of the present invention can obtain high sensitivity in a measurement method using any of a reaction format of a competitive method or a non-competitive method by using together with a second binding partner or a solid support on which the substance to be measured is immobilized.

Furthermore, the detection agent of the present invention can measure a subject substance with high sensitivity even in combination with any of solid supports commonly used in immunoassays, and extremely high-sensitivity measurement can be achieved particular in combination with a solid support having a particle shape such as magnetic particles.

### DESCRIPTION OF EMBODIMENTS

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. While the term “or” is used to mean “and/or” unless explicitly indicated to refer to alternatives only or the alternatives are mutually exclusive, it is herein used to mean both alternatives and “and/or.” When a numerical range is described using numerical values A and B, such as A to B, “A to B” is used to mean a numerical range of A or more and B or less, unless otherwise defined. Known methods and techniques, unless otherwise indicated, are performed by conventional methods well known in the art or by methods described in general references.

As used herein, the “measurement” includes, in addition to “measurement” with a general meaning of quantitatively or semi-quantitatively determining the amount of the substance to be measured, “detection” for determining the presence or absence of the substance to be measured.

The “binding partner” in the present invention is not particularly limited as long as it is a substance that can recognize and bind a substance to be measured using biological specificity and form a complex with the substance to be measured. Examples of binding using biological specificity include binding using antigen-antibody reaction, receptor-ligand reaction, enzyme-substrate reaction, protein-protein interaction (for example, reaction between IgG and protein A), protein-small molecule interaction (for example, reaction between avidin and biotin), protein-sugar chain interaction (for example, reaction between lectin and sugar chain), protein-nucleic acid interaction, nucleic acid hybridization reaction, or the like. For example, when using antigen-antibody reaction as biologically specific reaction, the combination of the substance to be measured and the binding partner is a combination of an antigen (substance to be measured) and an antibody (binding partner), or a combination of an antibody (substance to be measured) and an antigen (binding partner). When using enzyme-substrate reaction as biologically specific reaction, the combination of the substance to be measured and the binding partner is a combination of an enzyme (substance to be measured) and a substrate (binding partner), or a combination of a substrate (substance to be measured) and an enzyme (binding partner).

When the “second binding partner” is used in the present invention in addition to the “first binding partner”, the second binding partner is not particularly limited as long as it is a substance that can recognize and bind a substance to be measured using biological specificity, and can form a complex with the substance to be measured, and a substance that can bind to the substance to be measured in a region that does not overlap with a region to which the first binding partner binds. The “first binding partner” and the “second binding partner” may be the same or different substances. Usually, a portion of the substance to be measured to which the second binding partner can bind is different from a portion to which the first binding partner can bind, and the second binding partner is a different substance from the first binding partner at least in the portion or ability to bind to the substance to be measured. However, when the substance to be measured has a plurality of portions to which the first binding partner can bind, the second binding partner may be the same substance as the first binding partner, and the second binding partner can bind to the substance to be measured at a portion to which the first binding partner does not bind. Further, the binding of the second binding partner to the substance to be measured may use the same biologically specific reaction as the binding of the first binding partner to the substance to be measured, or may use a different biological reaction. For example, as a combination of first binding partner-substance to be measured-second binding partner, combinations of antibody (first binding partner)-antigen (substance to be measured)-antibody (second binding partner) or antigen (first binding partner)-antibody (substance to be measured)-antibody (second binding partner) and the like can be used utilizing only antigen-antibody reaction. Alternatively, using antigen-antibody reaction and enzyme-substrate reaction, combinations of antibody (first binding partner)-enzyme (substance to be measured)-substrate (second binding partner) or enzyme (first binding partner)-substrate (substance to be measured)-antibody (second binding partner) and the like can also be used.

As the binding partner of the present invention, an antibody or antigen that can bind to a substance to be measured using an antigen-antibody reaction having extremely high specificity and high binding affinity among biologically specific reactions is preferable. Furthermore, an antibody is more preferable since a new binding partner can be prepared for a substance to be measured in which a specific binding partner does not exist in nature.

The “antibody” used as the binding partner of the present invention may not necessarily maintain the entire structure of an immunoglobulin molecule as long as it can show sufficient specificity and affinity for the substance to be measured, and may be an antigen-binding fragment of the antibody. The antigen binding capacity of the antibody is governed by a variable region of the antibody, and a constant region of the antibody may not necessarily be present. Therefore, as the “antibody” of the present invention, in addition to five types of immunoglobulin molecules (IgG, IgM, IgA, IgD, IgE), Fab, Fab′, F(ab′)₂, Fd obtained by removing V_{L} from Fab, single-chain Fv fragment (scFv) and a diabody that is its dimer, a single domain antibody (sdAb) obtained by removing V_{L} from scFv or the like can be used, but it is not limited thereto.

The antibody of the present invention can be obtained commercially or can be prepared by a known standard method. When preparing an antibody against the substance to be measured, an experimental animal such as rabbit, mouse, rat, guinea pig, donkey, goat, sheep or chicken is immunized with the substance to be measured, and an antibody that specifically binds to the substance to be measured is produced in the animal body, and antisera or polyclonal antibody containing the antibody is prepared, or cells involved in antibody production can be fused with myeloma cells and then cloned to prepare a monoclonal antibody. Alternatively, an artificial antibody having a structure that is not produced in an animal body can be prepared in vitro by expressing a chemically synthesized antibody gene in E. coli or the like by a genetic engineering technique.

When an antigen-binding fragment is used as the antibody of the present invention, it can be obtained by enzymatic digestion of the antibody prepared as described above by a known method. Fab is obtained by decomposition with papain, F(ab′)₂ is obtained by treatment with pepsin, and Fab′ is obtained by reducing F(ab′)₂. Alternatively, scFv can be prepared by linking a heavy chain variable region (V_{H}) and a light chain variable region (V_{L}) of the antibody with a linker peptide with high mobility by genetic operation.

The “subject substance” that can be measured by the present invention may be any substance as long as there is a binding partner capable of binding to it using biological specificity, and examples thereof include, but are not limited to, proteins (such as antigens, antibodies, receptors, enzymes, and lectins), peptides, sugar chains (sugar chains such as monosaccharides, oligosaccharides, and polysaccharides), lipids, nucleic acids, low molecular compounds, hormones (such as steroid hormones, amine hormones, and peptide hormones), tumor markers, allergic substances, pesticides, environmental hormones, drugs of abuse, viruses, or cells (such as bacteria and blood cells), and the like.

The sample containing the substance to be measured to be subjected to the measurement according to the present invention includes almost all liquid samples including, in addition to blood (whole blood, plasma, serum), lymph, saliva, urine, stool, sweat, mucous, tears, perfusion, nasal discharge, neck or vaginal secretions, semen, pleural fluid, amniotic fluid, ascites, middle ear fluid, synovial fluid, gastric aspirate and biological fluids such as extracts and disrupted fluids of tissues and cells, foods, soil, and solutions such as extracts and disrupted liquids of plants, river water, hot spring water, drinking water, contaminated water, and the like.

When a subject substance is measured by a competitive method using the detection agent of the present invention, the substance to be measured needs to be previously immobilized on a solid support. The “solid support on which the substance to be measured is previously immobilized” of the present invention refers to a solid support on which the substance to be measured that binds to the first binding partner competitively with the substance to be measured released in the liquid sample is previously immobilized. The substance to be measured previously immobilized on the solid support may not necessarily retain exactly the same three-dimensional structure as the substance to be measured present in the liquid sample. The substance to be measured immobilized on the solid support may be exactly the same substance as the substance to be measured released in the liquid sample as long as the structure capable of binding to the first binding partner using biological specificity is retained in the state immobilized on the solid support, or may be a fragment thereof, or further may be a substance linked to a polymer compound (for example, protein) serving as a carrier.

The “reporter substance” of the present invention is not particularly limited as long as it can generate a signal that can be quantitatively measured, and any substance can be used. Examples thereof include radioisotopes, enzymes, fluorescent substances, luminescent substances, and the like. When using a radioisotope, a fluorescent substance or a luminescent substance as the reporter substance, radiation, fluorescence or luminescence generated by the substance can be quantitatively measured as a signal. When the reporter substance is an enzyme, an appropriate substrate is allowed to act, and color, fluorescence or luminescence derived from the finally produced dye, fluorescent substance or luminescent substance is measured as a signal. As the reporter substance of the present invention, any of the above substances can be used. However, an enzyme that can increase the amount of a reaction product by adding an excess of the substrate, and can amplify the final signal is preferable.

In recent years, a method called a cycling method has been developed as a method for amplifying a signal from a reporter substance. In this method, a luminescent substance whose structure has been changed by luminescence or a reaction product by enzyme reaction is converted into a structure before luminescence or into a substrate state of the enzyme by redox reaction, whereby a luminescent substance or an enzyme reaction product can be produced repeatedly, and it is possible to amplify a signal from the luminescent substance or the enzyme reaction product. The redox reaction can be carried out using a chemically inert electrode, or also can be accelerated by redox enzyme. As the reporter substance of the present invention, an enzyme which generates an electrochemically active luminescent substance or an electrochemically active substance as a reaction product, that can be combined with a cycling method, is preferable. A more preferable reporter substance is an enzyme which can achieve higher sensitivity by combining signal amplification by addition of an excess of the substrate and signal amplification by a cycling method as described above, and it can be used as an enzyme reaction product together with an appropriate substrate which generates an electrochemically active substance.

Radioisotopes that can be used as the reporter substance of the present invention include ³H, ¹²⁵I, and the like. The fluorescent substances include fluorescein and its derivatives (for example, FITC), tetramethylrhodamine (TAMRA) and its derivatives (for example, TRITC), Cy3, Cy5, Texas Red, phycoerythrin (PE), quantum dots (Quantum dot, trade name Qdot (registered trademark)) and the like, but are not limited thereto. The luminescent substances include luminol derivatives (for example, isoluminol), acridinium derivatives (for example, acridinium ester), aequorin, ruthenium complexes (for example, divalent ruthenium pyridine complexes) and the like, but are not limited thereto. In particular, a divalent ruthenium pyridine complex can be reconverted to a structure before luminescence via a trivalent complex by the cycling method and can amplify a signal, thus is a preferable luminescent substance as the reporter substance of the present invention.

When an enzyme is used as the reporter substance of the present invention, the enzyme activity can be measured by a method such as a colorimetric method, a fluorescence method or a luminescence method, by combining the enzyme with an appropriate substrate. The enzymes of the present invention include horseradish peroxidase (HRP), β-galactosidase (β-GAL), alkaline phosphatase (ALP), glucose oxidase (GOD), luciferase, aequorin and the like, but are not limited thereto. For example, HRP can detect the activity by a colorimetric method using 1,2-phenylenediamine or 3,3′,5,5′-tetramethylbenzidine as a substrate, and can detect the activity by a fluorescence method using 4-hydroxyphenylacetic acid or 3-(4-hydroxyphenyl) propionic acid as a substrate and a luminescence method using luminol as a substrate. β-GAL can detect the activity by a colorimetric method using 2-nitrophenyl-β-D-galactopyranoside as a substrate, a fluorescence method using 4-methylumbelliferyl-β-D-galactopyranoside as a substrate, and a luminescence method using AMPGD that is an adamantyl 1,2-dioxetane derivative as a substrate. ALP can detect the activity by a colorimetric method using 4-nitrophenyl phosphate as a substrate, a fluorescence method using 4-methylumbelliferyl phosphate as a substrate, and a luminescence method using AMPPD that is an adamantyl 1,2-dioxetane derivative as a substrate. In addition, when the enzyme activity is detected by a luminescence method, not only luminescence from the product generated by the enzyme reaction is detected directly, but also luminescence generated by exciting a luminescent substance by the enzyme reaction product can be detected. For example, luminescence generated by reacting an indoxyl derivative as a substrate with ALP or β-GAL and reacting the generated hydrogen peroxide with isoluminol can be also measured.

Furthermore, the enzyme as the reporter substance can reuse the reaction product by combining with the cycling method, and can further enhance the signal from the reaction product. For example, NAD+ generated by using ALP as an enzyme and allowing a substrate, NADP, to act thereon can be reconverted to NAD+ via NADH by a cycling method. By combining circular reaction by this cycling method with a reaction that produces a formazan dye by redox reaction, production of formazan dye is amplified, making it possible to measure the enzyme activity of ALP with extremely high sensitivity despite a colorimetric method, and making it possible to determine the concentration of a very small amount of a substance to be measured.

The “gold nanoparticles” of the present invention refer to nano-sized gold fine particles, and refer to particles capable of binding a first binding partner and a plurality of reporter substances to the surface of the gold nanoparticles. The gold nanoparticles of the present invention form a detection agent with the first binding partner and the plurality of reporter substances. When one of the first binding partners contained in the detection agent binds to the substance to be measured, the detection agent contains a plurality of reporter substances, thus the plurality of reporter substances are indirectly bound to one substance to be measured. While only one reporter substance can bind to one substance to be measured in a general labeled immunoassay, a plurality of reporter substances can be bound to one substance to be measured according to the detection agent of the present invention. Therefore, it is possible to markedly amplify a signal from the reporter substance correlated with the amount of the substance to be measured.

The gold nanoparticles of the present invention have an average particle diameter in the range of about 1 to 400 nm, preferably about 10 to 200 nm, and more preferably about 20 to 150 nm. When the average particle diameter is in the range of 20 nm to 150 nm, a very good result with a high signal-to-noise ratio (S/N ratio) at the time of measurement has been obtained (see Examples below), and gold nanoparticles having any average particle diameter in the same range can also be preferably used. Here, also in consideration of ease of handling of a colloidal gold solution containing gold nanoparticles, particularly preferred aspect includes gold nanoparticles with an average particle diameter in the range of 20 nm or more and less than 100 nm, and particularly 40 nm or more and 80 nm or less. On the other hand, when the average particle diameter is in the range of 80 nm or more and 150 nm or less, a relatively strong signal can be obtained even when the concentration of the substance to be measured is low. Therefore, depending on the substance to be measured, another particularly preferred aspect includes gold nanoparticles with an average particle diameter in the range of 80 nm or more and 150 nm or less, particularly 100 nm or more and 150 nm or less.

In the method of the present invention, particularly when high sensitivity and high accuracy are required, in the case of gold colloid, it is also possible to increase the number of colloidal particles having the same shape, for example, spherical particles, or uniformize the particle size of colloidal particles, for example, form colloid in which so-called particle size distribution curve is narrowed (particle size distribution is narrow), by concentrating the particle size at one point of a specific particle size such as 40 nm, 80 nm, or 120 nm. The degree of dispersion of the particle diameter can be evaluated by a polydispersity index (PDI), and a particle group with a narrow particle size distribution can be prepared by known techniques so that the PDI value is 0.1 or less, more preferably 0.07 or less, and further preferably 0.05 or less as indexes.

The average particle diameter can be usually measured by a dynamic light scattering method. For example, in the case of gold nanoparticles, the particle size distribution of the colloidal gold liquid in which the particles are dispersed can be measured by determining the average particle size after measuring with a dynamic light scattering particle size distribution analyzer. The shape of the particles is not particularly limited, and may be various shapes such as a sphere, a shell, a rod, a rice, a pyramid, a prism, a star, and a plate, and spherical gold nanoparticles are particularly preferable in that many first binding partners can be immobilized on the surface of the particles so as to be able to bind to the substance to be measured without steric hindrance.

The gold nanoparticles of the present invention can be produced by a known method, and can be produced by a chemical method such as reduction of gold halide or a physical method such as laser ablation. Examples of the chemical method include a method of reducing a tetrachlorogold(III) salt (H[AuCl₄]) solution in the presence of a reducing agent such as citric acid to produce seed particles, and then slowly growing the particles under acidic conditions in the presence of a reducing agent such as ascorbic acid, and the like. According to this method, a colloidal gold solution containing uniform spherical gold nanoparticles having a desired average particle diameter in the range of about 10 to 200 nm can be produced.

The phrase “the first binding partner is directly immobilized on the gold nanoparticles” in the present invention refers that the first binding partner binds to and is immobilized on the gold nanoparticles with random orientation, without interposing a substance that binds to the first binding partner using biological specificity. The binding using biological specificity is binding as already described herein, for example, antigen-antibody reaction, receptor-ligand reaction, enzyme-substrate reaction, protein-protein interaction (for example, reaction between IgG and protein A), protein-small molecule interaction (for example, reaction between avidin and biotin), and the like. Examples of the substance that binds to the first binding partner using biological specificity include, for example, when the first binding partner is an antibody, in addition to antigens, antibodies that bind to Fc portion of the antibody, and antibody binding proteins such as protein A, protein G and protein L, which are bacteria-derived proteins. In the present invention, the first binding partner is immobilized on the gold nanoparticles without interposing the above-mentioned substance.

In one aspect of the invention, the first binding partner is passively adsorbed and immobilized based on electrostatic and/or hydrophobic interactions that occur with the surface of the gold nanoparticles. The surface of the colloidal gold particles is negatively charged in a buffer with a pH of about 6 to 8, so that the first binding partner composed of a protein such as an antibody can be easily immobilized. In another aspect, the surface of gold nanoparticles is chemically modified with functional groups such as amino groups, carboxyl groups and N-hydroxysuccinimide (NHS) groups, and the first binding partner is covalently bonded to these functional groups to be immobilized on the surface of gold nanoparticles. Since these functional groups bind to an arbitrary carboxyl group or amino group of the first binding partner, the first binding partner is immobilized on the surface of gold nanoparticle with random orientation. The functional group that modifies the surface of gold nanoparticles may bind to the gold nanoparticles via a spacer that does not show biologically specific interaction with the first binding partner such as polyethylene glycol (PEG). The spacer can be several kDa in size, for example, 1 to 5 kDa in size.

The reporter substance of the present invention can be directly immobilized on the gold nanoparticles, like the first binding partner, or the first binding partner can be labeled with the reporter substance, and the labeled first binding partner can be directly immobilized on the gold nanoparticles. Alternatively, the first binding partner is directly immobilized on the gold nanoparticles, and then the first binding partner can be labeled with a reporter substance. However, it is preferable to previously label the binding partner with the reporter substance and directly immobilize the labeled binding partner on the gold nanoparticles, from reasons that immobilization by passive adsorption is difficult when the reporter substance is other than a protein, binding capacity of the binding partner with a substance to be measured may be impaired by reacting the reporter substance with the binding partner immobilized on the surface of gold nanoparticles, it is necessary to immobilize a predetermined number of binding partners and a reporter substance on the surface of gold nanoparticles and supply a detection agent having a stable quality, and the like.

The labeling of the first binding partner with the reporter substance of the present invention can be performed according to a known standard method used when labeling low molecular weight antigens, high molecular weight antigens or antibodies by various reporter substances such as enzymes, fluorescent substances or luminescent substances. The reporter substance of the present invention binds at least 2 molecules, preferably 5, 10, 100, and more preferably more molecules to the gold nanoparticles. By directly or indirectly “plurally” binding the reporter substance to the gold nanoparticles to form a binder, the number of reporter substances that bind to the substance to be measured via the first binding partner included in the binder can be increased, and it is possible for the first time to amplify the signal intensity correlated with the amount of the substance to be measured. The binding of many reporter substances of the present invention to the gold nanoparticles can be easily achieved by passively adsorbing the gold nanoparticles by using an excess of the first binding partner labeled with the reporter substance, or by covalently binding the first binding partner labeled with the reporter substance using gold nanoparticles whose surface is chemically modified with a number of functional groups.

As the “solid support” in the present invention, those having various shapes can be used as long as a second binding partner or a substance to be measured can be immobilized, they can be subjected to B/F separation operation for removing a free substance to be measured that does not bind to the immobilized second binding partner or substance to be measured or the binder, and they are made of an inert material that does not affect binding using biological specificity. Examples of the solid support include small test tubes or microplates made of glass or plastic, plastic beads, magnetic particles, porous membranes where capillary phenomenon is caused by moisture in the sample, or microfluidic chips provided with flow channels in a small piece of glass or plastic and the like, but are not limited thereto, and any known solid supports generally used in immunoassays can be used in the present invention. Immobilization of the second binding partner or the substance to be measured on these solid supports can be performed by a standard method used in immunoassays. For example, it can be immobilized with a covalent bond by chemically modifying the solid support surface with a functional group or the like, or more generally, it can be immobilized passively by using a property of adsorbing the second binding partner or the substance to be measured to the solid support. The solid support on which the second binding partner or the substance to be measured is immobilized may be subjected to blocking treatment, for the purpose of suppressing nonspecific binding other than biologically specific binding to the second binding partner or the substance to be measured. In the blocking treatment, a known blocking agent (skim milk, casein, bovine serum albumin (BSA), gelatin, normal serum, or the like) is used to coat the solid support surface to which the second binding partner or the substance to be measured is not bound.

As the solid support of the present invention, magnetic particles are preferable, from reasons that the second binding partner or the substance to be measured immobilized thereon can efficiently form a binding pair in a liquid phase, recovery from the liquid phase by applying a magnetic field with a magnet or the like is possible and B/F separation can be easily performed, and the like. Furthermore, when amplifying a signal by a cycling method using a chemically inert electrode, magnetic particles are used as the solid support, thereby facilitating collection of the magnetic particles on the electrode surface by applying a magnetic field from a back face of the electrode. When a complex composed of the substance to be measured and the detection agent formed on the magnetic particles contains a luminescent substance or an enzyme as the reporter substance,

a luminescent substance or an enzyme reaction product is efficiently oxidized or reduced on the electrode surface and the cycling reaction is promoted, so that

the use of magnetic particles as the solid support also has an advantage that signal amplification is further accelerated.

The magnetic particles used in the present invention refer to particles with magnetism, and any particles can be used as long as the particles can be dispersed or suspended in a liquid phase, and can be separated from a dispersion or suspension by applying a magnetic field. The type of the particles is not particularly limited, and includes organic particles or inorganic particles (including metal particles), or particles composed of a combination of organic and inorganic particles. The magnetic particles in the present invention are particularly preferably those in which a magnetic body is contained inside organic (polymer) particles, further, it is more preferable that the magnetic body is contained only inside the particles and is not exposed on the particle surface. The magnetic body may be any of ferromagnetic, paramagnetic and superparamagnetic, but is preferably superparamagnetic because separation by a magnetic field and redispersion after removing the magnetic field are facilitated. Examples of the magnetic body of the present invention include metals such as iron, cobalt, manganese, chromium or nickel, alloys of the metals, or salts, oxides, borides or sulfides of the metals, rare earth elements having high magnetic susceptibility (for example, hematite or ferrite), and the like. Among them, iron oxide and ferrite are preferable from the viewpoint of safety, and magnetite (Fe₃O₄) is particularly preferable.

The size of the magnetic particles in the present invention is not particularly limited, and may be any of nanoparticles, microparticles, or milliparticles, but is preferably nanoparticles or microparticles. Among them, the magnetic particles in the present invention are preferably particles with an average particle diameter of 0.05 μm to 20 μm, more preferably particles with an average particle diameter of 0.1 μm to 10 μm, further preferably particles with an average particle diameter of 0.3 μm to 3 μm, and most preferably particles with an average particle diameter of 1.5 μm to 3 μm. Further, in the present invention, in order to obtain a good measurement signal, it is possible to define the size of the magnetic particles also preferable from the viewpoint of relation with size of the gold nanoparticles used. In comparison of the average particle diameter, the size of the magnetic particles is preferably in the range of 10 to 150 times, more preferably in the range of 15 to 75 times, and further preferably in the range of 15 to 50 times the size of the gold nanoparticles. As a specific combination regarding the size of the magnetic particles and the gold nanoparticles, for magnetic particles with an average particle diameter of 1.5 μm, combination use of gold nanoparticles with an average particle diameter of 20 nm to 150 nm, 40 nm to 150 nm, 40 nm to less than 100 nm, 40 nm to 80 nm, or 80 nm to less than 100 nm, preferably gold nanoparticles with an average particle diameter of 40 nm, 60 nm or 80 nm, or for magnetic particles with an average particle diameter of 3 μm, combination use of gold nanoparticles with an average particle diameter of 20 nm to 150 nm, 40 nm to 150 nm, 40 nm to less than 100 nm, 40 nm to 80 nm, or 80 nm to less than 100 nm, and preferably gold nanoparticles with an average particle diameter of 40 nm, 60 nm or 80 nm can be illustrated. Also, the average particle diameter of the magnetic body contained in the magnetic particles is preferably 0.1 to 10 nm, more preferably 0.5 to 5 nm, and further preferably 1 to 3 nm. When the average particle diameter of the magnetic body exceeds 10 nm, influence of residual magnetization appears, and even after the magnetic field is removed, mutual coupling between the magnetic particles remains and affects redispersibility of the particles, which is not preferable. The shape of the magnetic particles and the magnetic body contained in the magnetic particles in the present invention may be of any shape and does not necessarily have to be perfect spherical, but the magnetic particles are preferably spherical in that many second binding partners or substances to be measured can be immobilized on the surface of the magnetic particles without impairing their binding capacity due to steric hindrance.

The method of immobilizing the second binding partner or the substance to be measured on the magnetic particles of the present invention includes passive adsorption to the surface of the magnetic particles using an excess of the second binding partners or the substances to be measured. Alternatively, magnetic particles whose surface is modified with a protein capable of forming a biologically specific binding pair such as a functional group such as a carboxyl group or a tosyl group, an antibody, protein A or avidin are also commercially available, and using the particles, the second binding partner or the substance to be measured can also be immobilized by covalent bonding or biological interaction. As the immobilization method on magnetic particles, a method via a functional group or a protein is preferable because binding stability is higher than passive adsorption, and immobilization using a tosyl group has less steric hindrance as compared to immobilization via a protein and does not require treatment with a condensing agent as in the case of binding via a carboxyl group or the like, thus it is particularly preferable because the possibility of impairing binding activity of the second binding partner or the substance to be measured is small.

When magnetic particles are used as the solid support of the present invention, a reaction between the binder of the present invention and the second binding partner or the substance to be measured immobilized on the magnetic particles is performed in a liquid phase in a container generally used in an immunoassay, such as a test tube, a microplate or a microfluidic device. Then, the magnetic particles are collected by applying a magnetic field from the outside of the container, and are subjected to a washing operation for B/F separation, or a signal generated from the reporter substance is measured at a predetermined position. When using a microplate, a porous membrane, and a microfluidic chip as the solid support of the present invention, a liquid sample is directly added to these solid supports, and the second binding partner or the substance to be measured immobilized at a predetermined position on the solid supports forms a complex with the detection agent, and the signal from the reporter substance contained in the complex is measured.

In one aspect of the present invention, the detection agent composed of the first binding partner of the present invention, a plurality of reporter substances and gold nanoparticles is combined with a device composed of a solid support including a complex forming part on which the second binding partner or the substance to be measured is immobilized, and is provided as a kit for measuring the substance to be measured. In this aspect, the detection agent is added to the device at the same time as or subsequent to the liquid sample containing the substance to be measured, and forms a complex with the second binding partner or the substance to be measured in the complex forming part of the device. Such device can be produced using a solid support such as a microplate, a magnetic particle, a porous membrane, or a microfluidic chip. The complex forming part can be designed in any size or shape. For example, when the solid support is a microplate, the entire bottom surface of each well can be used as the complex forming part, and when the solid support is a magnetic particle, the entire surface of the particle can be used as the complex forming part.

In another aspect of the present invention, the detection agent of the present invention is provided in a form held in a detection agent holding part provided on a device composed of a solid support provided with a complex forming part and is incorporated in the device. The detection agent holding part is provided between a site on the device to which a liquid sample is added and the complex forming part, and the detection agent is dried and held in the detection agent holding part. When the liquid sample containing the substance to be measured added to the device passes through the detection agent holding part of the device, the detection agent dissolves in the liquid sample, and the detection agent can bind to the substance to be measured. A device capable of providing such a detection agent holding part can be produced using a solid support such as a porous membrane or a microfluidic chip.

In still another aspect of the present invention, a device is formed of a microfluidic chip, and instead of including a complex forming part, the microfluidic chip has a complex forming agent holding part and a complex capturing part. In the complex forming agent holding part located upstream of the complex capturing part, a complex forming agent is dried and held, and when a liquid sample containing the substance to be measured is added to the device and passes through the complex forming agent holding part, the complex forming agent dissolves in the liquid sample. The complex forming agent is composed of magnetic particles on which the second binding partner or the substance to be measured is immobilized, and forms a complex with the detection agent of the present invention in the liquid sample. The complex capturing part captures a complex forming agent containing the magnetic particles by applying a magnetic field. The magnetic field can be applied to the complex capturing part of the device by means such as providing a magnet in the complex capturing part of the device or placing a magnet outside the complex capturing part of the device. The magnet used here may be a permanent magnet or an electromagnet. The reporter substance contained in the detection agent captured in the complex capturing part generates a signal there. Since the amount of the reporter substance correlates with the amount of the substance to be measured, the amount of the substance to be measured can be determined by measuring the signal intensity from the reporter substance. The complex capturing part may have an electrode for a cycling reaction as necessary. The device having the complex forming agent holding part and the complex capturing part can be also provided as a kit together with the detection agent of the present invention, and a detection agent holding part may be further formed on the device. When forming the detection agent holding part in addition to the complex forming agent holding part on the device, these two holding parts may be provided at different positions upstream of the complex capturing part or at the same position. For example, the complex forming agent and the detection agent are previously mixed, and dried and held, whereby the complex forming agent holding part and the detection agent holding part can be provided at the same position.

A further aspect of the device formed by a microfluidic chip includes a device having only a complex capturing part, and this device can be provided as a kit in combination with a complex forming agent. As described above, the detection agent of the present invention can be included in a kit as a further component, or a detection agent holding part is provided on the device and the detection agent can be held in the detection agent holding part.

Microfluidic chips can be manufactured by methods known in the art, for example, can be manufactured by preparing a flow channel having a mixing section and a reaction section, one or more inlets, and a waste liquid storage section, on a small piece of glass or plastic. The inlet is used for injecting a liquid sample containing the substance to be measured, and an inlet for injecting a washing liquid and/or an enzyme substrate solution into the channel may be provided separately, as necessary.

The present invention further provides an immunoassay system in which a kit including the detection agent of the present invention and a measurement apparatus capable of measuring a signal generated from a reporter substance of the detection agent are combined. The present invention also provides an immunoassay system in which a device having the detection agent of the present invention in a detection agent holding part is combined with a measurement apparatus capable of measuring a signal generated from a reporter substance of the detection agent. The measurement apparatus has at least a device mounting part and a signal detection part. The device mounting part is designed so that a device which is a further component of the kit including the detection agent or a device having the detection agent holding part can be attached and detached. These devices have either a complex forming part or a complex capturing part that can hold a complex composed of the substance to be measured and the detection agent, and when the device is attached to the device mounting part of the measurement apparatus, it becomes possible to measure a signal generated from the reporter substance contained in the complex held in the complex forming part or the complex capturing part by the signal detection part of the measurement apparatus. As described above, since the signal generated according to the type of the reporter substance is different from fluorescence, luminescence, color, and radiation, a known detector corresponding to the generated signal is provided in the signal detection part of the measurement apparatus. Further, the measurement apparatus may be provided with a liquid feed pump as necessary, in order to control the speed at which the liquid sample containing the substance to be measured, the washing liquid, or the enzyme substrate solution moves on the device. Moreover, when combined with a device composed of a microfluidic chip, the measurement apparatus can also include a magnet for applying a magnetic field to the complex capturing part, a power supply for applying a voltage to electrodes of the complex capturing part, or the like.

The measurement of subject substance by the competitive method using the detection agent of the present invention can be performed, for example, as follows. When the substance to be measured is an antigen and the first binding partner is an antibody specific thereto, first, a certain amount of the antigen is immobilized on a solid support in a state where the three-dimensional structure of the antibody binding site is retained. The detection agent of the present invention is added to the immobilized antigen simultaneously with or after the addition of the antigen released in the liquid sample, and the antibody contained in the detection agent is reacted with the immobilized antigen. Since the binding between the antibody contained in the detection agent and the immobilized antigen is competitively inhibited by the free antigen, the amount of detection agent capable of forming a complex with the immobilized antigen depending on the amount of the free antigen in the liquid sample is reduced, and the amount of signal from the reporter substance contained in the detection agent is reduced. A competitive reaction was performed using liquid samples with known antigen concentration to create a standard curve (dose-effect curve), and a signal intensity measured when a liquid sample containing an unknown concentration of antigen was added is inserted into the standard curve, whereby the amount of antigen in the liquid sample can be determined.

When a subject substance is measured by sandwich assay using the detection agent of the present invention, the measurement can be performed, for example, as follows. When the substance to be measured is an antigen, as the second binding partner, a certain excess amount of the antibody is immobilized on a solid support using an antibody specific to the second binding partner. The free antigen in the liquid sample and the detection agent of the present invention are added to the immobilized antibody to form a complex composed of the immobilized antibody-antigen-detection agent, and the order of adding the antigen and the detection agent may be any of the following. First, only the antigen may be added and captured by the immobilized antibody, and then the detection agent may be added and reacted with the captured antigen (forward assay), the antigen and the detection agent may be reacted to form an antigen-detection agent complex, and then the antigen-detection agent complex may be added and captured by an immobilized antibody (reverse assay), or the antigen and the detection agent may be added simultaneously, and both may be simultaneously reacted with the immobilized antibody to form an immobilized antibody-antigen-detection agent complex (simultaneous assay). The immobilized antibody-antigen-detection agent complex formed on the solid support is separated from the free antigen and/or the detection agent by washing operation, and a signal intensity from the reporter substance contained in the detection agent in the complex is measured. In a sandwich assay, the reporter substance is captured on the solid support depending on the amount of free antigen contained in the liquid sample, so that the amount of antigen in the liquid sample can be determined by measuring the signal intensity from the reporter substance.

Hereinbelow, the present invention will be described more specifically with reference to examples. However, the present invention is not limited to these examples by any means.

### EXAMPLES

### Example 1

### Production of Gold Nanoparticles Immobilized with Enzyme-Labeled Antibody

Colloidal gold solution containing gold nanoparticles with an average particle diameter of any of 20 nm, 40 nm, 60 nm, 80 nm, 100 nm and 150 nm measured by a dynamic light scattering method (gold concentration (ICP) about 65 to 68 ppm, TANAKA KIKINZOKU KOGYO K. K.) were used as materials. For gold nanoparticles with an average particle diameter of 20 nm, 40 nm, 60 nm, 80 nm, 100 nm and 150 nm, PDI values indicating the degree of dispersion of the particle diameter were 0.080, 0.056, 0.061, 0.034, 0.022 and 0.020, respectively. To 9 mL of each colloidal gold solution, 1 mL of 100 mM tris buffer (pH 8.5) was added thereto and mixed. Further, 0.05 mL of 1.0 mg/mL solution of an alkaline phosphatase (ALP)-labeled anti-cardiac troponin I antibody using a phosphate buffer (pH 7.0) as a solvent was added, mixed, and reacted at 5° C. for 5 minutes. 0.1 mL of distilled water containing 10% bovine serum albumin (BSA) was added thereto, mixed and reacted at 5° C. for 5 minutes. The mixture was centrifuged at 12,000 g for 5 minutes, a supernatant was removed, 10 mL of phosphate buffered saline (PBS) containing 1% BSA was added, and the mixture was completely dispersed using an ultrasonic washer. The mixture was centrifuged again at 12,000 g for 5 minutes, a supernatant was removed, 1 mL of PBS containing 1% BSA was added, and the mixture was completely dispersed using an ultrasonic washer.

### Example 2

### Measurement of Cardiac Troponin I (cTnI) Using Microplate

(1) Preparation of Microplate Immobilized with cTnI

Into a 96-well microplate (manufactured by Nunc), an anti-cTnI antibody solution of 0.01 mg/mL concentration using a carbonate buffer (pH 9.5) as a solvent was dispensed in 0.1 mL portions, and the mixture was allowed to stand at 5° C. overnight. After removing the solution by suction, the microplate was washed three times with PBS. PBS containing 1% BSA was dispensed in 300 μL portions into each well and allowed to stand at 37° C. for 1 hour. After removing the solution by suction, the microplate was washed three times with PBS containing 0.05% (v/v) Tween20 (PBS-T).

(2) Measurement of cTnI

A cTnI solution at a concentration of 0 ng/mL, 1 ng/mL, 10 ng/mL, or 100 ng/mL diluted using a phosphate buffer (pH 7.0) as a solvent, and a colloidal gold solution with an average particle diameter of 80 nm prepared in Example 1 were each dispensed in 10 μL portions into each well, and reacted at room temperature for 5 minutes. After removing the solution by suction, the microplate was washed three times with PBS-T. BluePhos (manufactured by KPL), which is an ALP substrate solution, was dispensed in 100 μL portions into each well. After 10 minutes, a supernatant of each well was measured for absorbance at 600 nm using a commercially available plate reader.

The results are shown in Tables 1 and 2 below. Table 1 shows the absorbance at 600 nm corresponding to each antigen concentration. Table 2 shows S/N ratios when the absorbance at an antigen concentration of 0 ng/mL is defined as noise (N), and the absorbance at an antigen concentration of 1 ng/mL, 10 ng/mL, and 100 ng/mL is defined as signal (S).

### Example 3

### Measurement of Cardiac Troponin I (cTnI) Using Magnetic Particles

(1) Preparation of Magnetic Particles Immobilized with Anti-cTnI Antibody

2 mL of particle dispersion of magnetic particles with an average particle diameter of 1.5 μm and having a surface chemically modified with a tosyl group (trade name: MagnosphereTM MS160/Tosyl, manufactured by JSR Corporation) having a solid content concentration of 10 mg/mL was taken in a microtube, and the particles were collected using a magnet, then a supernatant was removed. 2 mL of 100 mM borate buffer (pH 9.5) was added thereto and mixed. An antibody solution containing 20 μg of anti-cTnl antibody diluted using a phosphate buffer (pH 7.0) as a solvent was added thereto and mixed, and 1 mL of 100 mM borate buffer (pH 9.5) containing 3 M ammonium sulfate was further added thereto and mixed. The mixture was reacted at room temperature for 24 hours while being gently inverted and mixed with a rotator. 0.02 mL of distilled water containing 10% bovine serum albumin (BSA) was added thereto and mixed, and the mixture was reacted at room temperature for 6 hours or more while being gently inverted and mixed with a rotator. The particles were collected with a magnet, and a supernatant was removed. A washing operation such as adding 5 mL of PBS-T thereto and mixing, collecting particles with a magnet, and removing the supernatant was repeated three times. 10 mL of PBS containing 1% BSA was added to the washed magnetic particles and mixed to disperse the particles.

(2) Measurement of cTnI

In a 96-well microplate, a cTnI solution at a concentration of 0 ng/mL, 1 ng/mL, 10 ng/mL, or 100 ng/mL, the magnetic particle dispersion prepared in the above (1), and a colloidal gold solution with an average particle diameter of 80 nm prepared in Example 1 were each dispensed in 10 μL portions into each well, and reacted at room temperature for 5 minutes. The particles were collected with a magnet plate for a 96-well microplate, and after removing the supernatant, the particles were washed three times with PBS-T. BluePhos (manufactured by KPL), which is an ALP substrate solution, was dispensed in 100 μL portions into each well. After 10 minutes, a supernatant of each well was measured for absorbance at 600 nm using a commercially available plate reader.

The results are shown in Tables 1 and 2 below.

### Comparative Example 1

In the measurement of cTnl using the microplate of Example 2, an ALP-labeled antibody not immobilized on gold nanoparticles was used, instead of using the colloidal gold solution on which the ALP-labeled antibody prepared in Example 1 was immobilized. The concentration of the ALP-labeled antibody was adjusted to be the same as the concentration of the ALP-labeled antibody immobilized on the gold nanoparticles contained in the colloidal gold solution (OD₅₀=6.0) used in Example 2, and dispensed in 10 μL portions into each well, and the mixture was reacted.

The results are shown in Tables 1 and 2 below.

### Comparative Example 2

In the measurement of cTnI using the magnetic particles of Example 3, an ALP-labeled antibody not immobilized on gold nanoparticles was used, instead of using the colloidal gold solution on which the ALP-labeled antibody prepared in Example 1 was immobilized. The concentration of the ALP-labeled antibody was adjusted to be the same as the concentration of the ALP-labeled antibody immobilized on the gold nanoparticles contained in the colloidal gold solution (OD₅₅₀=6.0) used in Example 3, and dispensed in 10 μL portions into each well, and the mixture was reacted.

The results are shown in Tables 1 and 2 below.

TABLE 1

| Antigen | | | | |
|---|---|---|---|---|
| concen- | Abs. 600 | | | |
| tration | | | Comparative | Comparative |
| (ng/mL) | Example2 | Example3-1 | Example1 | Example2 |
| 100 | 0.947 | 2.023 | 0.105 | 0.381 |
| 10 | 0.232 | 0.584 | 0.042 | 0.187 |
| 1 | 0.082 | 0.187 | 0.030 | 0.114 |
| 0 | 0.067 | 0.106 | 0.030 | 0.111 |

The numerical values of the absorbance of Example 3-1 listed in Table 1 were obtained by experiments performed on the same day as Example 2 and Comparative Examples 1 and 2.

TABLE 2

| Antigen | | | | |
|---|---|---|---|---|
| concen- | S/N | | | |
| tration | | | Comparative | Comprative |
| (ng/mL) | Example2 | Example3-1 | Example1 | Example2 |
| 100 | 14.13 | 19.08 | 3.50 | 3.43 |
| 10 | 3.46 | 5.51 | 1.40 | 1.68 |
| 1 | 1.22 | 1.76 | 1.00 | 1.03 |
| 0 | — | — | — | — |

(Note that when the antigen concentration is 0 (ng/mL), no signal is included, and thus the S/N ratio is not shown in the table. The same applies to the following tables.)

According to the results shown in Tables 1 and 2, it has been clarified that a value almost same as the S/N ratio obtained when the cTnl antigen at a concentration of 10 ng/mL or 100 ng/mL is measured using only the enzyme-labeled antibody (Comparative Example 1 or 2) is obtained when the cTnl antigen at a concentration of 1 ng/mL or 10 ng/mL is measured using the detection agent in which the enzyme-labeled antibody is immobilized on the gold nanoparticles (Example 2 or 3-1). By using the detection agent in which the enzyme-labeled antibody is immobilized on the gold nanoparticles, the number of labeled enzymes that indirectly bind to the cTn I antigen could be increased, and the signal from the labeled enzyme that binds to the cTnI antigen could be amplified. As a result, it was possible to increase the sensitivity of the measurement by about 10 times. Amplification effect of the signal by the detection agent in which the enzyme-labeled antibody was immobilized on the gold nanoparticles was exerted regardless of the type of the solid support capturing the antigen, and even higher sensitivity could be obtained when the solid support was the magnetic particles (Example 3-1) more than the microplate (Example 2).

### Comparative Example 3

In the measurement of cTnI using the magnetic particles of Example 3, a suspension of latex particles on which the ALP-labeled antibody was immobilized was used, instead of using the colloidal gold solution on which the ALP-labeled antibody prepared in Example 1 was immobilized. Latex particles with an average particle diameter of 75 nm (manufactured by Merck) and 1 μm (manufactured by Polysciences) were used. The immobilization of the ALP-labeled anti-cTnl antibody on the latex particles was performed by passive adsorption according to the immobilization method on the gold nanoparticles described in Example 1. The concentration of the suspension of latex particles was adjusted so that the concentration of the ALP-labeled antibody immobilized on the latex particles be the same as the concentration of the ALP-labeled antibody immobilized on the gold nanoparticles contained in the colloidal gold solution (OD₅₅₀=6.0) used in Example 3, and dispensed in 10 μL portions into each well, and the mixture was reacted.

The results are shown in Tables 3 and 4 below. Table 3 shows the absorbance at 600 nm corresponding to each antigen concentration. Table 4 shows S/N ratios when the absorbance at an antigen concentration of 0 ng/mL is defined as noise (N), and the absorbance at an antigen concentration of 1 ng/mL, 10 ng/mL, and 100 ng/mL is defined as signal (S).

TABLE 3

| Antigen | Abs. 600 | | |
|---|---|---|---|
| concen- | Example3-2 | Comparative Example3 | |
| tration | Average particle | Average particle | Average particle |
| (ng/mL) | diameter of 80 nm | diameter of 75 nm | diameter of 1 μm |
| 100 | 1.227 | 0.470 | 0.248 |
| 10 | 0.254 | 0.239 | 0.145 |
| 1 | 0.180 | 0.227 | 0.131 |
| 0 | 0.127 | 0.206 | 0.123 |

The numerical values of the absorbance of Example 3-2 listed in Table 3 were obtained by experiments performed on the same day as Comparative Example 3.

TABLE 4

| Antigen | S/N | | |
|---|---|---|---|
| concen- | Example3-2 | Comparative example3 | |
| tration | Average particle | Average particle | Average particle |
| (ng/mL) | diameter of 80 nm | diameter of 75 nm | diameter of 1 μm |
| 100 | 9.66 | 2.28 | 2.02 |
| 10 | 2.00 | 1.16 | 1.18 |
| 1 | 1.42 | 1.10 | 1.07 |
| 0 | — | — | — |

According to the results shown in Tables 3 and 4, it has been clarified that a value almost same as the S/N ratio obtained when the cTnI antigen at a concentration of 10 ng/mL or 100 ng/mL is measured using the enzyme-labeled antibody immobilized on the latex particles is obtained when the cTnI antigen at a concentration of 1 ng/mL or 10 ng/mL is measured using the detection agent in which the enzyme-labeled antibody is immobilized on the gold nanoparticles. The detection agent in which the enzyme-labeled antibody is immobilized on the gold nanoparticles can amplify the signal from the labeled enzyme about 10 times as compared to the enzyme-labeled antibody immobilized on the latex particles, and markedly increased the sensitivity of measurement of subject substance.

### Example 4

### Examination of Average Particle Diameter of Gold Nanoparticles

CTnI was measured using magnetic particles in the same manner as in Example 3, using the colloidal gold solutions with an average particle diameter of 20 nm, 40 nm, 60 nm, 80 nm, 100 nm and 150 nm prepared in Example 1.

The results are shown in Tables 5 and 6 below. Table 5 shows the absorbance at 600 nm corresponding to each antigen concentration. Table 6 shows S/N ratios when the absorbance at an antigen concentration of 0 ng/mL is defined as noise (N), and the absorbance at an antigen concentration of 1 ng/mL, 10 ng/mL, and 100 ng/mL is defined as signal (S).

TABLE 5

| | Abs. 600 | | | | | |
|---|---|---|---|---|---|---|
| | Average | Average | Average | Average | Average | Average |
| Antigen | particle | particle | particle | particle | particle | particle |
| concentration | diameter of | diameter of | diameter of | diameter of | diameter of | diameter of |
| (ng/mL) | 20 nm | 40 nm | 60 nm | 80 nm | 100 nm | 150 nm |
| 100 | 1.999 | 2.026 | 1.949 | 1.730 | 2.333 | 2.240 |
| 10 | 0.375 | 0.461 | 0.494 | 0.565 | 1.371 | 1.153 |
| 1 | 0.127 | 0.232 | 0.224 | 0.256 | 0.321 | 0.227 |
| 0 | 0.126 | 0.204 | 0.203 | 0.202 | 0.163 | 0.128 |

TABLE 6

| | S/N | | | | | |
|---|---|---|---|---|---|---|
| | Average | Average | Average | Average | Average | Average |
| Antigen | particle | particle | particle | particle | particle | particle |
| concentration | diameter of | diameter of | diameter of | diameter of | diameter of | diameter of |
| (ng/mL) | 20 nm | 40 nm | 60 nm | 80 nm | 100 nm | 150 nm |
| 100 | 15.87 | 9.93 | 9.60 | 8.56 | 14.31 | 17.50 |
| 10 | 2.98 | 2.26 | 2.43 | 2.80 | 8.41 | 9.01 |
| 1 | 1.01 | 1.14 | 1.10 | 1.27 | 1.97 | 1.77 |
| 0 | — | — | — | — | — | — |

According to the results shown in Tables 5 and 6, it has been clarified that a signal of sufficient intensity is obtained even in a measurement system using any gold nanoparticles with an average particle diameter in the range of 20 nm to 150 nm. In addition, in a measurement system with an antigen concentration of 10 ng/mL or more, it has been clarified that the measurement system using gold nanoparticles with an average particle diameter in the range of 20 nm to 150 nm is a measurement system with a sufficiently high S/N ratio, such that the S/N ratio greatly exceeds 2 in any case. From these results, it can be understood that not only when using gold nanoparticles with an average particle diameter of 80 nm, but also when using gold nanoparticles with any average particle diameter in the range of 20 nm to 150 nm, the labeled antibody is immobilized using the gold nanoparticles to form a binder, thereby producing a good signal amplification effect.

### Example 5

### Examination of Average Particle Diameter of Magnetic Particles and Method of Immobilizing Antibody

According to the method described in Example 3 (1) “Preparation of Magnetic Particles Immobilized with Anti-cTnI Antibody”, using a particle dispersion of magnetic particles with an average particle diameter of 3 μm and having a surface chemically modified with a tosyl group (trade name: Magnosphere™ MS300/Tosyl, manufactured by JSR Corporation), magnetic particles having an anti-cTnI antibody immobilized on the surface were prepared. Furthermore, using a particle dispersion of biologically modified magnetic particles with an average particle diameter of 0.3 μm or 2.6 μm and having streptavidin immobilized on the surface (trade name: Estapor, manufactured by Merck) and a biotin-labeled anti-cTnI antibody, magnetic particles having an anti-cTnI antibody immobilized on the surface were prepared.

Using these magnetic particles, cTnI was measured using the colloidal gold solution with an average particle diameter of 80 nm prepared in Example 1, according to the method described in Example 3 (2) “Measurement of cTnI”.

The results are shown in Tables 7 and 8 below. Table 7 shows the absorbance at 600 nm corresponding to each antigen concentration. Table 8 shows S/N ratios when the absorbance at an antigen concentration of 0 ng/mL is defined as noise (N), and the absorbance at an antigen concentration of 1 ng/mL, 10 ng/mL, and 100 ng/mL is defined as signal (S).

TABLE 7

| | Abs. 600 | | | |
|---|---|---|---|---|
| Antigen | Chemically modified | | Biologically modified | |
| concentration | Average particle | Average particle | Average particle | Average particle |
| (ng/mL) | diameter of 1.5 μm | diameter of 3 μm | diameter of 0.3 μm | diameter of 2.6 μm |
| 100 | 1.730 | 1.015 | 0.237 | 0.662 |
| 10 | 0.565 | 0.301 | 0.129 | 0.187 |
| 1 | 0.256 | 0.139 | 0.139 | 0.157 |
| 0 | 0.202 | 0.130 | 0.111 | 0.117 |

TABLE 8

| | S/N | | | |
|---|---|---|---|---|
| Antigen | Chemically modified | | Biologically modified | |
| concentration | Average particle of | Average particleof | Average particle of | Average particle of |
| (ng/mL) | diameter1.5 μm | diameter3 μm | diameter0.3 μm | diameter2.6 μm |
| 100 | 8.56 | 7.81 | 2.14 | 5.66 |
| 10 | 2.80 | 2.32 | 1.16 | 1.60 |
| 1 | 1.27 | 1.07 | 1.25 | 1.34 |
| 0 | — | — | — | — |

According to the results of Tables 1 and 2, magnetic particles are suitable as the solid support used for measurement in combination with the detection agent of the present invention. Further, according to the results shown in Tables 7 and 8, as the method for immobilizing an antibody on magnetic particles, it has been clarified that a method of covalently binding an antibody using a functional group such as a tosyl group is more suitable than binding using biological specificity of avidin-biotin and the like. Furthermore, it has been found that when using magnetic particles with a size of about 15 to 50 times the average particle diameter of the gold nanoparticles, a particularly excellent signal amplification effect is exhibited, and the highest measurement sensitivity is obtained.

### INDUSTRIAL APPLICABILITY

The present invention provides a detection agent capable of markedly increasing the number of reporter substances that indirectly bind to a substance to be measured by immobilizing a binding partner that specifically recognizes the substance to be measured on the surface of gold nanoparticle together with a plurality of reporter substances, and significantly amplifying a signal correlated with the amount of the substance to be measured. According to the detection agent of the present invention, since the subject substance can be measured with higher sensitivity, the present invention is particularly useful in the industrial field in which a very small amount of subject substance needs to be specifically measured with high sensitivity, and quickly and simply, and has industrial applicability not only in the field of clinical chemical test, but also in fields such as food inspection and environmental analysis.

1. A method for measuring a subject substance, comprising:
(i) forming a complex containing a substance to be measured, and a detection agent composed of a first binding partner that specifically recognizes the substance to be measured, a plurality of reporter substances and gold nanoparticles, and
(ii) measuring signals from the reporter substances contained in the complex, wherein
the first binding partner is directly immobilized on the gold nanoparticles in the detection agent and is labeled with the reporter substance.

2. The method according to claim 1, wherein the complex further contains a second binding partner that specifically recognizes the substance to be measured, which is immobilized on a solid support.

3. The method according to claim 1, wherein the complex contains the substance to be measured immobilized on a solid support.

4. The method according to claim 1, wherein
the (i) is a process of contacting a liquid sample containing the substance to be measured, with the detection agent composed of a first binding partner that specifically recognizes the substance to be measured, a plurality of reporter substances and gold nanoparticles to form a complex containing the substance to be measured and the detection agent.

5. The method according to claim 4, wherein
the process (i), simultaneously with or after the contact, further comprises a process of contacting a solid support on which a second binding partner that specifically recognizes a substance to be measured is previously immobilized, and
the (ii) is a process of measuring signals from the reporter substances contained in the complex formed on the solid support.

6. The method according to claim 4, wherein
the process (i) is a process performed in a reaction system containing a solid support on which the substance to be measured is previously immobilized, and
the (ii) is a process of measuring signals from the reporter substances contained in the complex formed on the solid support.

7. The method according to claim 1, wherein
the (i) is a process of using at least a liquid sample containing the substance to be measured, the detection agent composed of the first binding partner that specifically recognizes the substance to be measured, a plurality of reporter substances and gold nanoparticles, and a solid support on which a second binding partner that specifically recognizes the substance to be measured is previously immobilized, and is
a process of forming a complex containing the substance to be measured and the detection agent by contacting the solid support on which the second binding partner is previously immobilized, with the liquid sample containing the substance to be measured, subsequently contacting the detection agent composed of the first binding partner that specifically recognizes the substance to be measured and the plurality of reporter substances and the gold nanoparticles, and
the (ii) is a process of measuring signals from the reporter substances contained in the complex formed on the solid support.

8. The method according to claim 2, wherein the solid support is selected from the group consisting of a microplate, a magnetic particle, a porous membrane, and a microfluidic chip.

9. The method according to claim 8, wherein the solid support is a magnetic particle.

10. The method according to claim 9, wherein the magnetic particles have an average particle diameter of 0.3 to 3 μm.

11. The method according to claim 1, wherein the gold nanoparticles have an average particle diameter of 20 to 150 nm.

12. The method according to claim 1, wherein the binding partner is an antigen or an antibody or an antigen-binding fragment thereof.

13. (canceled)

14. The method according to claim 1, wherein the reporter substance is selected from the group consisting of a radioisotope, an enzyme, a fluorescent substance, and a luminescent substance.

15. The method according to claim 1, wherein the reporter substance is an electrochemically active luminescent substance or an enzyme that generates an electrochemically active substance as a reaction product.

16. The method according to claim 4, wherein the liquid sample is a biological fluid.

17. A detection agent for measuring a subject substance, comprising
a first binding partner that specifically recognizes a substance to be measured, a plurality of reporter substances and gold nanoparticles, wherein
the first binding partner is directly immobilized on the gold nanoparticle and is labeled with the reporter substance, and
the reporter substance can generate a signal with an intensity correlated with the amount of the substance to be measured bound to the first binding partner.

18. The detection agent according to claim 17, wherein the gold nanoparticles have an average particle diameter of 20 to 150 nm.

19. The detection agent according to claim 17, wherein the binding partner is an antigen or an antibody or an antigen-binding fragment thereof.

20. (canceled)

21. The detection agent according to claim 17, wherein the reporter substance is selected from the group consisting of a radioisotope, an enzyme, a fluorescent substance, and a luminescent substance.

22. (canceled)

23. The detection agent according to claim 17, wherein the reporter substance is directly immobilized on the first binding partner.

24-36. (canceled)
